(19) United States
(12) Patent Application Publication
(10) Pub. No.: US 2005/0191678 A1
(43) Pub. Date: Sep. 1, 2005

(54) Genetic predictability for acquiring a disease or condition

(75) Inventors: Gilles Lapointe, Mississauga (CA); Louis Perusse, Sillery (CA)

Correspondence Address: SIMPSON & SIMPSON, PLLC, 5555 MAIN STREET, WILLIAMSVILLE, NY, 14221-5406, US

(73) Assignee: GeneOb USA Inc., Amherst, NY (US)

(21) Appl. No.: 11/056,047

(22) Filed: Feb. 11, 2005

Related U.S. Application Data
(60) Provisional application No. 60/544,087, filed on Feb. 12, 2004.

(51) Int. Cl. C12Q 1/68; G06F 19/00; G01N 33/48; G01N 33/50

(52) U.S. Cl. 435/6; 702/20

(57) ABSTRACT

A method for assessing susceptibility of a subject to a genetically related disease or condition relative to a general population. The method includes the steps of: determining the presence or absence of a plurality of risk factors associated with the subject and having a correlation with the disease or condition; assigning a risk score, to each of the selected risk factors determined to be present, based upon a strength of correlation assigned to the factor with respect to the disease or condition; and combining the risk scores to calculate an overall susceptibility score, wherein the overall susceptibility score represents susceptibility of the subject to the disease or condition in relation to a base score representing the risk that a member of the general population will have the disease or condition without consideration of risk factors. The risk factors require the inclusion of at least two of age, gender, race, and family history and require the inclusion of a plurality of polymorphisms selected for known correlation with the disease or condition.

### CROSS REFERENCE TO RELATED APPLICATIONS

Priority is claimed from U.S. Provisional Application 60/544,087 filed Feb. 12, 2004.

### BACKGROUND OF THE INVENTION

This invention relates to a method for the utilization of risk factors for assessing the probability that an individual (subject) will acquire a disease or condition. Otherwise stated, the invention relates to a method for assessing susceptibility of a subject to the disease or condition.

It has been known that certain risk factors are correlated or associated with particular diseases or conditions and can be used to assess probability that an individual will acquire the disease or condition based upon whether or not the individual has the risk factor. For example it is well established that a history of smoking increases the probability that an individual will acquire lung cancer to the extent that about 87% of lung cancers occur in individuals with a history of exposure to smoking and that about one in ten smokers will get lung cancer compared with about one in 100 for non-smokers, i.e. a risk for smokers about 10 times higher for smokers than non-smokers. It is also known that exposure of an individual to heavy concentrations of airborne asbestos particles also increases the probability that an individual will develop lung cancer at a rate about 7 times higher than the population in general. It is further known that a combination of risk factors, e.g. smoking and exposure to asbestos, further increases the probability of acquiring lung cancer, e.g. an asbestos worker who smokes is 50 to 90 times more likely to develop lung cancer than the population as a whole, a multiplication of the risks together (data from the National Institute of Health and the American Cancer Society).

Among common risk factors associated with numerous diseases and conditions are age, gender, ethnicity (including race), and obesity. Certain risk factors, e.g. obesity, are themselves acquired conditions subject to assessment using other risk factors, e.g. sedentary life style, high calorie diet, etc.

Recently it has been found that certain genetic polymorphisms (alterations in nucleic acid structure of genes from gene structures usually encountered) can have a correlation with certain diseases and conditions. Examples of such diseases and conditions that may have correlating polymorphisms include obesity; certain cancers, e.g. the BRCA1 gene associated with certain breast cancers; schizophrenia; rheumatoid arthritis; asthma; lupus; hypertension; diabetes; macular degeneration, e.g. an SNP of manganese superoxide dismutase gene; and heart disease.

It has further been noted that the occurrence of multiple diseases or condition associated polymorphisms may increase the risk that the disease or condition may be acquired, e.g. as described in published PCT Application WO 02/102980 A2, incorporated herein by reference as background art. Neither this reference, nor others known to the inventors herein, discuss or suggest how multiple polymorphisms may be treated as risk factors for consideration in conjunction with other risk factors, such as age, gender, ethnicity (including race), and family history for assessing susceptibility of a subject to a genetically related disease or condition. This is unfortunate since up to now the effects of multiple risk factors, in addition to the presence of a plurality of disease or condition related polymorphisms, has not been considered.

Among the conditions that is itself a risk factor for many other diseases and conditions is obesity. The World Health Organization states that an escalating global epidemic of overweight (25<BMI<30) and obesity (BMI≧30) is taking over many parts of the world. The number of obese adults increased 67 percent between 1995 and 2000 worldwide. Up to 1.7 billion people worldwide are overweight or obese, making it the biggest health threat facing the world's population. In the seven major markets (United States, France, Germany, Italy, Spain, United Kingdom, and Japan) the number of obese adults has been estimated at 95 million in 2000. In the United States, in 2003, 120 million adults are overweight and 60 million are obese (64% of the adult population). Childhood obesity is also a well known fact, with over 15% of boys and girls above the percentile corresponding to adult BMI>25 in countries such as Hungary, France, Italy, Germany, etc. Again, the USA counts over 15% of its children as overweight and 15% as obese. In China, an estimated 200 million people could become obese in the next 10 years. In France, we now count 5.39 million obese and 20 million overweight or obese. The frequency of obese individuals, between 35-44 years of age, increased 51% in 6 years. After 45 years of age, overweight reaches nearly one men out of two and one quarter of women are overweight. Recent data state also that 64% of adult females and 36% of adult males are on a diet

The health consequences of obesity range from a number of non-fatal complaints that impact on the quality of life such as respiratory difficulties, musculo-skeletal problems, skin problems and infertility, to complaints that lead to an increased risk of premature death including non-insulin dependant diabetes, gallbladder disease, cardiovascular problems (hypertension, stroke and coronary heart disease) and cancers. In the United States alone 17 million people have been diagnosed with type II diabetes. An other 16 million people are in a pre-diabetes category where their blood sugar level is higher than normal. The way to diagnose diabetes is through the fasting glucose test, a biochemical analysis of the blood sugar content. There is no way, however' to test for predisposition. Obesity also brings heart complications. According to the American Heart Association, cardiovascular diseases claim 1 life every 33 seconds in the United States. In 1996, among United States adults, $31 billion in treatment costs for coronary vascular disease was related to overweight or obesity.

The increase in the prevalence of obesity observed worldwide in the past 50 years has occurred in a changing environment characterized by a progressive reduction in energy expenditure associated with physical activity and the abundance of highly palatable foods. These environmental changes occurred over a period of time that is too short to cause changes in the frequencies of genes associated with obesity. Thus, genes that were selected for energy storage in the primitive hunter/gatherer populations are now detrimental in an era of food abundance. From a genetic point of view, this suggests that gene-environment interactions are important in determining an individual's susceptibility to obesity and related metabolic complications. From an environmental point of view, this implies that the benefit of avoiding exposure to an environmental risk factor will be greater for individuals with a high-risk genotype than for those with a low-risk genotype.

Overweight and obesity result from an imbalance between the calories consumed and the calories used by the body. When the calories consumed exceed the calories burned, the body is in positive energy balance and over time weight gain will occur. The excess calories are stored in the fat cells. When the calories burned exceed the calories consumed, the body is in negative energy balance and over time weight loss will occur.

Based on data from the American Heart Association, about 680 Americans die each day of coronary heart disease. Heart disease is the number 1 killer in men and women in the United States. Major risk factors for heart disease include type II diabetes (or adult onset diabetes), high blood pressure and high blood cholesterol levels. These risk factors of heart disease are much more frequent in overweight and obese subjects than in subjects with a normal body weight. According to the American Diabetes Association, there are 16 million people with type II diabetes in the United States and about 90% of them are obese. Another 17 million has a condition called pre-diabetes, with higher than normal blood glucose levels, but not high enough for a diagnostic of type II diabetes. The risk of developing the medical conditions associated with obesity is not the same for every overweight or obese subject. Genes also play a role in determining this risk.

As previously discussed, several genotypes have already been identified in animals and human to cause disorders and physiological states. Others have been identified to be correlated with a disorder or a physiological state (Y. C. Chagnon, T. Rankinen, E. E. Snyder, S. J. Weisnagel, L. Pérusse, and C. Bouchard. 2003. Obesity Research 11:313-367, and E. E. Snyder, B. Walts, L. Pérusse, Y. C. Chagnon, J. Weisnagel, T. Rankinen, C. Bouchard. 2004. Obesity Research, 12:369-439.incorporated herein by reference as background art). There is now convincing evidence indicating a significant contribution of genetic factors for most obesity phenotypes (Roberts S B, Greenberg A S. Nutrition Reviews 1:41-49, 1996. Maes H H, Neale M C, Eaves L J. Behav Genet 27:325-351, 1997. Pérusse L, Chagnon Y C, Rice T, D. C. R, Bouchard C. Médecine Sciences 14:914-924, 1998. Pérusse L, Chagnon Y C, Bouchard C. In Update: surgery for the morbidly obese patient Deitel M, Cowan G S M, Eds. Toronto, F D-Communications Inc., 2000, p. 1-12. Pérusse L, Chagnon Y C, Bouchard C. In Genetics in endocrinology Baxtwer J D, Ed. Philadelphia, Pa., Lippincott Williams & Wilkins, 2002, p. 275-273-273).

The non-U.S. Patent references referred to in the backgraound of the invention are incorporated by reference as background art. The U.S. Patents referred to herein are incorporated by reference.

Although associations between individual disorders and individual genotypes are known, a need remains for a method of assessing the overall predisposition of a mammal, especially humans, to develop obesity, type II diabetes and obesity-related heart disease. The current invention satisfies is directed toward this need.

### BRIEF DESCRIPTION OF THE INVENTION

In accordance with the invention, a method is provided for assessing susceptibility of a subject to a genetically related disease or condition relative to a general population. The method includes the steps of:
determining the presence or absence of a plurality of risk factors associated with the subject and having a correlation with the disease or condition;
assigning a risk score, to each of the selected risk factors determined to be present, based upon a strength of correlation assigned to the factor with respect to the disease or condition; and
combining the risk scores to calculate an overall susceptibility score, wherein the overall susceptibility score represents susceptibility of the subject to the disease or condition in relation to a base score representing the risk that a member of the general population will have the disease or condition without consideration of risk factors.

The risk factors require the inclusion of at least two and preferably three of age, gender, race, and family history and require the inclusion of a plurality of polymorphisms selected for known correlation with the disease or condition.

Usually the risk score represents the risk that a subject will have the disease or condition, when the subject also has the risk factor, divided by the risk that a subject will have the disease or condition, when the subject does not have the risk factor. More particularly, a risk score may be determined from data concerning a series of groups a), b), c) and d) within the general population. Group a) is a group having both the risk factor and the disease or condition. Group b) has the risk factor and does not have the disease or condition. Group c) does not have the risk factor and has the disease or condition and group d) does not have the risk factor and does not have the disease or condition. The risk score may then be calculated from a risk ratio obtained from the formula [a/(a+b)][/c/(c+d)]. The risk ratio may be multiplied by a constant to obtain the risk score. The constant is chosen to place the risk score and base score in comparable units. If adjustment to obtain comparable units is not necessary, the constant is 1.

The method may be used for assessing relative susceptibility of a subject to obesity, obesity related diabetes, and obesity related heart disease by the steps of:
obtaining a biological sample containing genomic DNA from a subject;
testing the biological sample for nucleic acid polymorphism risk factors in one or both alleles of a gene, which polymorphisms each have a correlation with increased susceptibility to obesity, obesity related diabetes, or obesity related heart disease where the testing is for polymorphisms in at least three genes affecting the components of energy balance and in at least three genes associated with an increased risk of diabetes or heart disease in overweight and obese subjects;
assigning a risk score, to each of the selected polymorphism risk factors determined to be present, based upon a strength of correlation assigned to the factor with respect to the disease or condition; and
following the steps with respect to combining risk factors as described above.

### DETAILED DESCRIPTION OF THE INVENTION

A “polymorphism” in a gene is one of the alternative forms of a portion of the gene that are known to occur in the human population. For example, many genes are known to exhibit single nucleotide polymorphic forms where the identity of a single nucleotide residue of the gene differs among the forms. Each of the polymorphic forms represent a single polymorphism, as the term is used herein. Other known polymorphic forms include alternative forms in which multiple consecutive or closely spaced, non-consecutive nucleotide residues vary in sequence, forms which differ by the presence or absence of a single nucleotide residue or a small number nucleotide residues, and forms that exhibit different mRNA splicing patterns.

A “single nucleotide polymorphism” (“SNP”) is one of the alternative forms of a portion of a gene that vary only in the identity of a single nucleotide residue in that portion.

A “disorder associated polymorphism” is an alternative form of a portion of a gene where occurrence of the alternative form in the genome of a human has been correlated with exhibition in the human of a disease or condition.

A “non-disorder associated polymorphism” is an alternative form of a portion of a gene for which no significant correlation has been made between occurrence of the alternative form in the genome and a disease or condition.

“General population” means the entire population under consideration with respect to susceptibility to a disease or condition including those who have and do not have the disease or condition.

“Disease”, as used herein, means an impairment of physiological function having a genetic cause or correlation, whether or not it also has non-genetic causes or correlations.

“Condition”, as used herein, means a physiological manifestation that may include but does not necessarily include impairment of physiological function and that has a genetic cause or correlation, whether or not it also has non-genetic causes or correlations. In its broadest sense, condition includes and is generic to disease.

“Risk factors” are attributes of an individual or a group of individuals having a correlation to a disease or condition. Examples of risk factors are age, ethnicity including race, family history, gender, diet, exercise history, exposure to toxic or carcinogenic agents, and exposure to biological agents.

“Ethnicity” means ethnic heritage, i.e. heritage from a group having a sufficiently long history of sufficiently complete genetic isolation to obtain unique genetic characteristics.

“Race” means the traditional Negroid, Caucasian, Mongoloid and Australoid races and are included within “ethnicity”.

“Family history” means the presence of a disease or condition in a close relative, especially a parent, sibling, or child of a subject but may also include the presence of a disease or condition in a grandparent, aunt, uncle or first cousin.

“Risk score” is a number proportional to the strength of correlation of a risk factor to a disease or condition. The risk score is usually the risk that a subject will have the disease or condition when the subject has a risk factor, divided by the risk that the subject will have the disease or condition when the subject does not have the risk factor. A risk score for acquiring a disease or condition may also be presented as an increased percentage of risk over risk of an individual not having risk factors. In such a case the “Base score”, subsequently described, is 100.

“Combining the risk scores” means adding or multiplying risk scores to obtain a close approximation of probability that an individual (subject) will acquire a disease or condition. One method for such combining is to simply add the risk scores. Another method for combining is to multiply together the probable number of individuals per risk factor that will acquire the disease per a number in the population as whole, e.g. 6 per 1000 for risk factor a), 5 per 1000 per risk factor b) to obtain combined risk scores (overall susceptibility score) of 30 per 1000, i.e. (6×5=30).

“Base score” is the risk that an individual will acquire the disease or condition in the overall population without consideration of risk factors, e.g. 1 per 1000. The base score may thus be compared with the susceptibility score, e.g. 1 in 1000 as compared with the susceptibility score in the above example of 30 per 1000.

A “wild type” form of the polymorphism is the “usual” form of the gene found in the genome with no disease or pathological state associated.

“Obesity” relates to a chronic disease characterized by an excess amount of body fat. “Overweight and obesity” result from an imbalance between the calories consumed and the calories used by the body.

“Diabetes” is used to described the condition of high blood sugar content and refers to type II diabetes.

“Genotyping” relates to the methodology used in a laboratory to test for the presence or absence of a polymorphism. The technique can relate to DNA sequencing of the region of the chromosome carrying the polymorphism, the use of fluorescence or dyes in order to detect the presence or absence of the variation, or any other technique which can be used to detect the presence or absence of the polymorphisms.

“Sequence tagged site” (STS) is a short (200 to 500 base pair) DNA sequence that has a single occurrence in the human genome and whose location and base sequence are known.

“Restriction site polymorphism” is a DNA polymorphism in which one of the two nucleotide sequences contains a recognition site for a particular endonuclease but the second lacks such a site (restriction site). Also a site in a DNA segment in which bordering bases are vulnerable to restriction enzymes (cleavage site).

“Restriction fragment length polymorphism” (RFLP) is an intra species variation in the length of DNA fragments generated by action of restriction enzymes.

“BMI” is body mass index calculated by body weight in kilograms divided by height in meters squared. Body mass index is an indication of percent body fat.

The method of the invention may be used for assessing relative susceptibility of a human to genetic predisposition to obesity and to obesity related diabetes and heart disease. The method comprises assessing occurrence in the human's genome of variations (polymorphisms) in several genes. A listing of examples of such genes are as follows:
(a) genes affecting the 3 components of the energy balance:
   (i) calories consumed, i.e. regulation of appetite. Such genes include leptin receptor gene (LEPR), dopamine receptor D2 gene (DRD2), type 2C serotonin receptor gene (HTR2C), and melanocortin-4 receptor gene (MCR4);
   (ii) capacity of the fat cells to store the extra energy. Such genes include peroxisome proliferator activated receptor gamma-2 gene (PPARG), tumor necrosis factor alpha gene (TNFA), and fatty acid binding protein 2 gene (FABP2); and
   (iii) calories burned. Such genes include adrenergic receptor beta-2 gene (ADRB2), adrenergic receptor beta-3 gene (ADRB3), glucocorticoid receptor gene (GRL), uncoupling protein 2 gene (UCP2) and uncoupling protein 3 gene (UCP3),
(b) genes associated with an increased risk of heart disease in overweight and obese subjects: (these genes may also be associated with increased risk of diabetes, high blood pressure and high blood cholesterol).
   (i) genes associated with the risk of diabetes. Such genes include insulin receptor substrate-1 gene (IRS1), sulfonyl urea receptor 1 gene (SUR1), and calpain 10 gene (CAPN10);
   (ii) genes associated with high blood pressure. Such genes include angiotensin converting enzyme gene (ACE) and angiotensinogen gene (AGT); and
   (iii) genes associated with high blood cholesterol. Such genes include apolipoprotein E gene (APOE), apolipoprotein B gene (APOE), and lipoprotein lipase gene (LPL).

Occurrence of any of the specific polymorphisms in the genes from groups (a) and (b) is an indication that the human is more susceptible to obesity, and/or type II diabetes, and/or heart disease. Furthermore, occurrence of a plurality of the polymorphisms is an indication that the human is even more susceptible to obesity, and/or type II diabetes, and/or heart disease. The genes are selected from the group consisting of (a) and (b). In one embodiment, the method comprises assessing occurrence of variations in the 20 genes from the groups (a) and (b) which will give a complete overlook at the susceptibility to obesity and the susceptibility to heart disease in a human being, especially when considered in conjunction with other risk factors.

In one embodiment, the method comprises assessing occurrence of variations in genes selected from a combinasion of (a) and (b) in the human genome. The combinations allow the calculation of the susceptibility to diabetes only, or to heart disease only.

The method by which occurrence of an individual polymorphism is assessed is not critical and numerous methods are well known to those skilled in the art, e.g. as described in U.S. Pat. Nos. 5,869,242; 6,448,010; 6,602,662; 6,811,977; 6,825,009; 6,825,010; and 6,841,128 all of which are incorporated herein by reference. Other such techniques and methods are described in numerous peer reviewed publications, a few of which are: Sauer et al., Nucleic Acids Res. 28(5):e13-e13 (2000); Xu et al., Nucleic Acids Res. 31(8):e43-e43 (2003); Ross et al., J. Clin. Microbiol. 38(10):3581-3584 (2000); Matise et al., Am. J. Hum. Genet. 73(2):271-284 (2003); Olivier et al., Nucleic Acids Res. 30(12):e53-e53 (2002), which are hereby incorporated by reference as background art.

A method for the detection of polymorphisms can be as follows but is not restricted only to this method. Fluorescence polarization (FP) can be used as a detection method for the primer extension assay, when a dye-labeled dideoxy terminator is incorporated allele-specifically in the presence of a matching DNA template (See e.g. Chen, X., Levine, L., and Kwok, P.-Y. 1999. Fluorescence polarization in homogeneous nucleic acid analysis. Genome Res. 9: 492-498 incorporated by reference as background art.).

Two oligonucleotides (or primers) are chosen complementary to the sequence surronding the polymorphism site in order to synthesize a fragment which is amplified through polymerase chain reaction. This fragment is detected fy fluorescence using specific pairs of hybridization probes. These probes are oligonucleotide sequences complementary to the sequence adjacent to the polymorphism site. One probe is labeted at the 5′-end with a dye, a fluorophore. To avoid extension this probe is modified at the 3′-end by phosphorylation. The other probe is labeles at the 3′-end with an other fluorophore. During hybridization of the two probes and the amplified fragment of DNA, the probes come in close proximity, resulting in fluorescence resonance energy transfer. Light emission is measured with a fluorescence polarization reader. In such reaction the donor fluorophore is excited by the light source generated by the reader instrument. Part of the excitation energy is transferred to the acceptor fluorophore. The emitted fluorrescence of this fluorophorre is measured.

This detection method using fluorescence polarization is not only limited to primer extension. FP is also a detection method for the 5′-nuclease assay, where a fluorescent probe is cleaved during the polymerase chain reaction only when it is annealed to a perfectly complementary template (See e.g. Latif S, Bauer-Sardina I, Ranade K, Livak K J, Kwok P Y. Genome Res. 2001 Mar 1; 11(3): 436-440 incorporated by reference as background art).

Once the presence of polymorphism risk factors has been assessed, risk scores for susceptibility to obesity and/or diabetes, and/or heart disease can be calculated In another aspect, the invention relates to a method of selecting a diet and exercise program that would benefit a human. The method comprises assessing risk factors, including the occurrence in the human's genome of polymorphisms in genes as described above. After assessing occurrence of the polymorphisms, a diet and exercise program is tailored to the individual's needs.

Table 1 depicts an example of results that can be obtained by analyzing occurrence of polymorphisms in 20 genes. The black stars indicate the presence of a gene that affects a specific condition. For example, the gene ADRB2 has a strong impact [symbol] on the number of calories burned by the body, but has also a moderate impact [symbol] on the amount of body fatness and the risk of high blood pressure. The circles indicate the number of copies of the high-risk gene, the gene sequence carrying the variation increasing the susceptibility to obesity and/or diabetes and/or heart disease. A filled circle indicates the presence of one variation, two filled circle indicate the presence of two copies of thevariation.

The numbers to the right of the image indicate the risk score for each polymorphism calculated according to the impact of the polymorphism on a condition, the number of copies of thepolymorphism, and gender, age and ethnic origin. The numbers on the bottom of the image indicate the risk score for genetic susceptibility to obesity, and the risk score for genetic susceptibility to heart disease (0 being low risk and 10 a higher risk).

TABLE 1
Result of a hypothetical human DNA testing for obesity, diabetes and heart disease.

| | Susceptibility | | | Susceptibility to | | | | |
|---|---|---|---|---|---|---|---|---|
| | to obesity | | | heart disease | | | Result | |
| | Calories | Body | Calories | | Blood | Blood | | Risk |
| GENE | Consumed | fatness | Burned | Diabetes | pressure | cholesterol | Genotype | score |

Calories consumed

| | | | | | |
|---|---|---|---|---|---|
| LEPR | [symbol] | [symbol] | | ●● | 2.5 |
| DRD2 | [symbol] | [symbol] | [symbol] | ●◯ | 3.0 |
| HTR2C | [symbol] | | | ◯◯ | 0 |
| MC4R | [symbol] | [symbol] | | ◯◯ | 0 |

Body fatness

| | | | | | |
|---|---|---|---|---|---|
| PPARG | [symbol] | [symbol] | | ●◯ | 2.0 |
| TNFA | [symbol] | [symbol] | [symbol] | ●● | 3.0 |
| FABP2 | [symbol] | [symbol] | [symbol] | ◯◯ | 0 |

Calories burned

| | | | | | | | |
|---|---|---|---|---|---|---|---|
| ADRB2 | | [symbol] | [symbol] | | [symbol] | ●● | 4.0 |
| ADRB3 | | [symbol] | [symbol] | | [symbol] | ●● | 3.0 |
| GRL | [symbol] | [symbol] | [symbol] | | | ●◯ | 2.5 |
| UCP2 | | | [symbol] | [symbol] | | ●● | 3.5 |
| UCP3 | [symbol] | | [symbol] | | | ◯◯ | 0 |

Risk of diabetes

| | | | | |
|---|---|---|---|---|
| IRS1 | | [symbol] | ●● | 2.0 |
| SUR1 | | [symbol] | ◯◯ | 0 |
| CAPN10 | [symbol] | [symbol] | ◯◯ | 0 |

Risk of high blood pressure

| | | | | | |
|---|---|---|---|---|---|
| ACE | | [symbol] | [symbol] | ●● | 4.0 |
| AGT | [symbol] | | [symbol] | ◯◯ | 0 |

Risk of high blood cholesterol

| | | | | |
|---|---|---|---|---|
| APOE | [symbol] | [symbol] | ●◯ | 1.0 |
| APOB | [symbol] | [symbol] | ◯◯ | 0 |
| LPL | [symbol] | [symbol] | ◯◯ | 3.5 |

Obesity genetic susceptibility risk score: 3.9/10; Heart disease susceptibility risk score 2.0/10

The invention permits DNA tests and methods to be used in conjunction with other risk factors to determine susceptibility to a disease or condition. The method will permit assessment of susceptibility including risk factors involving genetic predisposition to obesity and to obesity related diabetes and heart disease. The methodology permits the use of testing of the presence of variations (polymorphisms) in genes associated with obesity, obesity related diabetes and heart disease. This testing of variations gives information on whether or not the genes are (a) homozygous for the disorder associated polymorphism at a genomic site; (b) heterozygous for disorder-associated and non-disorder-associated polymorphisms at that site; and (c) for non-disorder-associated polymorphisms at that site. Assessments of genomic polymorphism content can be used to help determine the likelihood that a human will develop obesity, obesity related diabetes and heart disease. Risk scores associated with a plurality of polymorphisms associated with a disease or condition such as obesity can be combined to give a stronger, more stringent likelihood that the disease or condition will be acquired. The invention includes a series of complex associations between gene variations, disorders, conditions, and variables which are discussed herein.

The invention relates to methods for assessing the predisposition to obesity, obesity related diabetes, and obesity related heart disease of a human by assessing occurrence in the human's genome of genetic polymorphisms that are associated with several conditions. The methods do not diagnose a disorder or a disease associated with a gene polymorphism. The method verifies the occurrence of particular polymorphisms in particular genes disclosed herein. Using two or more polymorphisms in particular genes, one can assess the susceptibility of a human to obesity, obesity related diabetes, and obesity related heart disease in conjunction with other risk factors. The disorder and/or condition has to be associated with the occurrence of a polymorphism chosen.

In accordance with the invention, other risk factors are taken into consideration in addition to the presence of polymorphisms, e.g. studies in peer reviewed publications have shown that risk of obesity is about 2 to 8 times higher in families of obese individuals than in the population at large, a risk that tends to increase with the severity of obesity.

The results obtained in the QFS (Quebec Family Study) database indicate heritability estimates of about 25%-40% for body composition, 40%-50% for phenotypes indexing fat distribution and between 50%-55% for abdominal fat assessed by CT scan (Pérusse L, Chagnon Y C, Rice T, D. C. R, Bouchard C. Médecine Sciences 14:914-924, 1998.).

There is also considerable evidence supporting a role for genetic factors for the various metabolic complications associated with obesity with heritability estimates in the range of 25% to 70% for plasma lipids and lipoproteins and for phenotypes related to plasma glucose and insulin metabolism.

Physical activity and the availability of high fat energy dense foods are reported as the two principal modifiable environmental type risk factors through which many of the external forces promoting the development of obesity act. These external forces are mainly accounted for by the socio-cultural changes that have contributed to the development of the obesigenic environment characterizing the modern societies in which we live.

This invention is the first to describe methods and risk factors for assessing a human's predisposition to develop obesity, type II diabetes, and obesity-related heart disease by using a panel of several genes in conjunction with other risk factors.

Occurrence of any of a number of particular polymorphisms in particular genes can be assayed in order to assess for susceptibility to obesity, diabetes and/or heart disease. A non-limiting table of such genes and a list of examples of such polymorphisms are as follows:

TABLE 2
Genes Correlated With Obesity

| Gene | | |
|---|---|---|
| Symbol | Name of the gene | Function of expressed protein |
| LEPR | Leptin receptor | Receptor of the hormone leptin. Signals the brain on the |
| | | amount of fat stored in the fat cells. |
| DRD2 | Dopamine receptor D2 | Dopamine and serotonin are substances found in the |
| HTR2C | Type 2C serotonin receptor | nerve tissue and involved in the control of appetite and |
| | | the desire to eat. They bind to specific receptors to exert |
| | | their effects. Most of the obesity drugs on the market are |
| | | appetite suppressants that affect these pathways. |
| MC4R | Melanocortin-4 receptor | Plays a key role in decreasing the desire to eat. |
| PPARG | Peroxisome proliferator | Control of the development of the fat cells in the body. |
| | activated receptor gamma-2 | |
| TNFA | Tumor necrosis factor alpha | Substance secreted by the fat cells associated with |
| | | increased risk of obesity and diabetes |
| FABP2 | Fatty acid binding protein 2 | Control of fat absorption in the intestine. |
| ADRB2 | Adrenergic receptor beta-2 | Receptor to hormones regulating the amount of energy |
| ADRB3 | Adrenergic receptor beta-3 | used by the body and the amount of fat stored in the fat |
| | | cells. |
| GRL | Glucocorticoid receptor | Receptor to the “stress” hormone cortisol, which is |
| | | associated with an increased desire to eat and a reduced |
| | | amount of calories used. |
| UCP2 | Uncoupling protein 2 | Proteins involved in the dissipation of excess calories as |
| UCP3 | Uncoupling protein 3 | heat. |
| IRS1 | Insulin receptor substrate-1 | Element in the insulin-signaling pathways. Mutations in |
| | | the gene play a role in determining susceptibility to traits |
| | | related to type 2 diabetes |
| SUR1 | Sulfonyl urea receptor 1 | Receptor located in cells of the pancreas secreting |
| | | insulin. Drugs to lower blood glucose are acting on this |
| | | receptor. |
| CAPN10 | Calpain 10 | Protein associated with increased risk of diabetes. |
| ACE | Angiotensin converting enzyme | Proteins involved in the production of a substance |
| AGT | Angiotensinogen | secreted by blood vessels and causing and increase in |
| | | blood pressure. |
| APOE | Apolipoprotein E | Proteins located on the surface of blood lipids and |
| APOB | Apolipoprotein B | controlling the production of the “good” and the “bad” |
| | | cholesterol in the blood. |
| LPL | Lipoprotein lipase | Protein involved in the degradation of triglycerides |
| | | circulating in the blood and thus control the amount of |
| | | fat taken up by the cells. |

Specific polymorphisms associated with obesity and obesity related diseases and are polymorphisms of the above genes are as follows:

A polymorphism manifested as a change from a glutamine residue to an arginine residue at amino acid residue 223 in leptin receptor protein encoded by exon 6 of leptin receptor gene (LEPR). This polymorphism is associated with obesity.

A polymorphism manifested as a change from a lysine residue to an arginine residue at amino acid residue 109 in leptin receptor protein encoded by leptin receptor gene (LEPR). This polymorphism is associated with obesity.

A polymorphism manifested as a change from a lysine residue to an asparagine residue at amino acid residue 656 in leptin receptor protein encoded by leptin receptor gene (LEPR). This polymorphism is associated with obesity.

A polymorphism manifested as a change from a serine residue to a serine residue at amino acid residue 343 in leptin receptor protein encoded by leptin receptor gene (LEPR) containing an altered codon. This polymorphism is associated with obesity.

A polymorphism manifested as a change from a serine residue to a cysteine residue at amino acid residue 311 in dopamine receptor protein encoded by dopamine receptor D2 gene (DRD2). This polymorphism is associated with obesity.

A polymorphism manifested at the Taq1A marker of dopamine receptor D2 gene (DRD2). This polymorphism is associated with obesity.

A polymorphism manifested as a change from a serine residue to a cysteine residue at amino acid residue 282 in dopamine receptor D2 protein encoded by dopamine receptor D2 gene (DRD2). This polymorphism is associated with obesity.

A polymorphism manifested at a NcoI RFLP (C→T exon 6) of dopamine receptor D2 gene (DRD2). This polymorphism is associated with obesity.

A polymorphism manifested as a change from a proline residue to a serine residue at amino acid residue 310 in dopamine receptor D2 protein encoded by dopamine receptor D2 gene (DRD2). This polymorphism is associated with obesity.

A polymorphism manifested as a change from a nucleotide alanine to a nucleotide guanine position demonstrated by SNP rs1124491(A/G) of dopamine receptor D2 gene (DRD2). This polymorphism is associated with obesity.

A polymorphism manifested as a change from a cysteine residue to a serine residue at amino acid residue 23 of type 2C serotonin receptor protein encoded by type 2C serotonin receptor gene (HTR2C). This polymorphism is associated with obesity.

A polymorphism manifested as a change from a leucine residue to a valine residue at amino acid residue 4 of type 2C serotonin receptor protein encoded by type 2C serotonin receptor gene (HTR2C). This polymorphism is associated with obesity.

A polymorphism manifested as a change from a proline residue to a glutamine residue at amino acid residue 83 of melanocortin-4 receptor protein encoded by melanocortin-4 receptor gene (MC4R). This polymorphism is associated with obesity. A polymorphism manifested as a change from a serine residue to a isoleucine residue at amino acid residue 169 of melanocortin-4 receptor protein encoded by melanocortin-4 receptor gene (MC4R). This polymorphism is associated with obesity.

A polymorphism manifested as a change from a isoleucine residue to a valine residue at amino acid residue 103 of melanocortin-4 receptor protein encoded by melanocortin-4 receptor gene (MC4R). This polymorphism is associated with obesity.

A polymorphism manifested as a change from an arginine residue to a glycine residue at amino acid residue 98 of melanocortin-4 receptor protein encoded by melanocortin-4 receptor gene (MC4R). This polymorphism is associated with obesity.

A polymorphism manifested as a change from a proline residue to an alanine residue at amino acid residue 12 of peroxisome proliferator activated receptor gamma-2 protein encoded by peroxisome proliferator activated receptor gamma-2 gene (PPARG). This polymorphism is associated with obesity. This polymorphism is associated with obesity related diabetes. This polymorphism is associated with obesity related diabetes.

A polymorphism manifested as a change from a proline residue to an alanine residue at amino acid residue 40 of peroxisome proliferator activated receptor gamma-2 protein encoded by peroxisome proliferator activated receptor gamma-2 gene (PPARG). This polymorphism is associated with obesity. This polymorphism is associated with obesity related diabetes.

A polymorphism manifested at position −308 changing a nucleotide guanine for a nucleotide alanine of tumor necrosis factor alpha gene (TNFA). This polymorphism is associated with obesity. This polymorphism is associated with obesity related heart disease. This polymorphism is associated with obesity related diabetes.

A polymorphism manifested as a change from a histidine residue to an asparagine residue at amino acid residue 52 of tumor necrosis factor alpha protein encoded by tumor necrosis factor alpha gene (TNFA). This polymorphism is associated with obesity. This polymorphism is associated with obesity related heart disease. This polymorphism is associated with obesity related diabetes.

A polymorphism manifested as a change from a proline residue to a leucine residue at amino acid residue 84 of tumor necrosis factor alpha gene (TNFA). This polymorphism is associated with obesity. This polymorphism is associated with obesity related heart disease. This polymorphism is associated with obesity related diabetes.

A polymorphism manifested as a change from an alanine residue to a threonine residue at amino acid residue 54 of fatty acid binding protein 2 encoded by fatty acid binding protein 2 gene (FABP2). This polymorphism is associated with obesity. This polymorphism is associated with obesity related diabetes.

A polymorphism manifested as a change from a threonine residue to an alanine residue at amino acid residue 55 of fatty acid binding protein 2 encoded by fatty acid binding protein 2 gene (FABP2). This polymorphism is associated with obesity. This polymorphism is associated with obesity related heart disease. This polymorphism is associated with obesity related diabetes.

A polymorphism demonstrated as SNP rs1511025. This polymorphism is associated with obesity, obesity related diabetes and obesity related heart disease.

A polymorphism manifested as a change from a glycine residue to an arginine residue at amino acid residue 16 of adrenergic receptor beta-2 protein encoded by adrenergic receptor beta-2 gene (ADBR2). This polymorphism is associated with obesity. This polymorphism is associated with obesity related heart disease.

A polymorphism manifested as a change from a glutamine residue to a glutamic acid residue at amino acid residue 27 of adrenergic receptor beta-2 protein encoded by adrenergic receptor beta-2 gene (ADBR2). This polymorphism is associated with obesity. This polymorphism is associated with obesity related heart disease.

A polymorphism manifested as a change from a threonine residue to an isoleucine residue at amino acid residue 164 of adrenergic receptor beta-2 protein encoded by adrenergic receptor beta-2 gene (ADBR2). This polymorphism is associated with obesity. This polymorphism is associated with obesity related heart disease.

A polymorphism manifested as a change from a serine residue to a cysteine residue at amino acid residue 220 of adrenergic receptor beta-2 protein encoded by adrenergic receptor beta-2 gene (ADBR2). This polymorphism is associated with obesity. This polymorphism is associated with obesity related heart disease.

A polymorphism manifested as a change from a tryptophan residue to an arginine residue at amino acid residue 64 of adrenergic receptor beta-3 protein encoded by adrenergic receptor beta-3 gene (ADRB3). This polymorphism is associated with obesity. This polymorphism is associated with obesity related heart disease.

A polymorphism manifested as a change from a threonine residue to a methionine residue at amino acid residue 265 of adrenergic receptor beta-3 protein encoded by adrenergic receptor beta-3 gene (ADRB3). This polymorphism is associated with obesity. This polymorphism is associated with obesity related heart disease.

A polymorphism manifested as a change from an asparagine residue to a serine residue at amino acid residue 363 of corticoid receptor protein encoded by corticoid receptor gene (GRL). This polymorphism is associated with obesity.

A polymorphism manifested as a change from a phenylalanine residue to a valine residue at amino acid residue 65 of corticoid receptor protein encoded by corticoid receptor gene (GRL). This polymorphism is associated with obesity.

A polymorphism manifested at position +647 of corticoid receptor protein encoded by corticoid receptor gene (GRL). This polymorphism is associated with obesity.

A polymorphism manifested as a change from an alanine residue to a valine residue at amino acid residue 55 of uncoupling protein 2 encoded by uncoupling protein 2 gene (UCP2). This polymorphism is associated with obesity. This polymorphism is associated with obesity related diabetes and obesity related heart disease.

A polymorphism manifested as a change from a nucleotide cytosine residue to a nucleotide thymine residue at position −55 of uncoupling protein 3 encoded by uncoupling protein 3 gene (UCP3). This polymorphism is associated with obesity.

A polymorphism manifested as a change from an arginine residue to a cysteine residue at amino acid residue 282 of uncoupling protein 3 encoded by uncoupling protein 3 gene (UCP3). This polymorphism is associated with obesity.

A polymorphism manifested as a change from a valine residue to a isoleucine residue at amino acid residue 102 of uncoupling protein 3 encoded by uncoupling protein 3 gene (UCP3). This polymorphism is associated with obesity.

A polymorphism manifested as a change from a tyrosine residue to a tyrosine residue at amino acid residue 99 of uncoupling protein 3 encoded by uncoupling protein 3 gene (UCP3) containing an altered codon. This polymorphism is associated with obesity.

A polymorphism manifested as a change from a methionine residue to a threonine residue at amino acid residue 209 of insulin receptor substrate-1 protein encoded by insulin receptor substrate-1 gene (IRS1). This polymorphism is associated with obesity and obesity related diabetes.

A polymorphism manifested as a change from a threonine residue to a threonine residue at amino acid residue 759 of sulfonyl urea receptor 1 protein encoded by sulfonyl urea receptor 1 gene (SUR1) containing an altered codon. This polymorphism is associated with obesity. This polymorphism is associated with obesity related heart disease. This polymorphism is associated with obesity related diabetes.

A polymorphism manifested as a change from an alanine residue to a serine residue at amino acid residue 1369 of sulfonyl urea receptor 1 protein encoded by sulfonyl urea receptor 1 gene (SUR1). This polymorphism is associated with obesity. This polymorphism is associated with obesity related heart disease. This polymorphism is associated with obesity related diabetes.

A polymorphism manifested as UCSNP-43 (g.4852 G/A) of (CAPN10). This polymorphism is associated with obesity. This polymorphism is associated with obesity related heart disease. This polymorphism is associated with obesity related diabetes.

A polymorphism manifested as UCSNP-44 (g.4841 T/C) of calpain 10 gene (CAPN10). This polymorphism is associated with obesity. This polymorphism is associated with obesity related heart disease. This polymorphism is associated with obesity related diabetes.

A polymorphism manifested as a change from a threonine residue to an alanine residue at amino acid residue 504 of calpain 10 protein encoded by calpain 10 gene (CAPN10). This polymorphism is associated with obesity. This polymorphism is associated with obesity related heart disease. This polymorphism is associated with obesity related diabetes.

A polymorphism manifested as an ACE I/D polymorphism of angiotensin converting enzyme gene (ACE). This polymorphism is associated with obesity. This polymorphism is associated with obesity related heart disease. This polymorphism is associated with obesity related diabetes.

A polymorphism manifested as a change from an arginine residue to a serine residue at amino acid residue 1286 of angiotensin converting enzyme encoded by angiotensin converting enzyme gene (ACE). This polymorphism is associated with obesity. This polymorphism is associated with obesity related heart disease. This polymorphism is associated with obesity related diabetes.

A polymorphism manifested as a change from a methionine residue to a threonine residue at amino acid residue 235 of angiotesinogen protein encoded by angiotensinogen gene (AGT). This polymorphism is associated with obesity. This polymorphism is associated with obesity related heart disease.

A polymorphism manifested as a change from a threonine residue to a methionine residue at amino acid residue 174 of angiotesinogen protein encoded by angiotensinogen gene (AGT). This polymorphism is associated with obesity. This polymorphism is associated with obesity related heart disease.

A polymorphism manifested by the isoforms ApoeE2, ApoeE3, or ApoeE4 of apolipoprotein E gene (APOE). This polymorphism is associated with obesity. This polymorphism is associated with obesity related heart disease.

A polymorphism manifested as a change from a cysteine residue to an arginine residue at amino acid residue 130 of apolipoprotein E encoded by apolipoprtein E gene (APOE). This polymorphism is associated with obesity. This polymorphism is associated with obesity related heart disease.

A polymorphism manifested by the EcoRI locus of apolipoprotein B gene (APOB). This polymorphism is associated with obesity. This polymorphism is associated with obesity related heart disease.

A polymorphism manifested as a change from an aspartic acid residue to an asparagine residue at amino acid residue 9 of lipoprotein lipase encoded by lipoprotein lipase gene (LPL). This polymorphism is associated with obesity. This polymorphism is associated with obesity related heart disease.

A polymorphism manifested as a truncated lipoprotein lipase at amino acid residue 446 due to a change from a serine code for amino acid residue 447 to a stop codon in lipoprotein lipase gene (LPL). This polymorphism is associated with obesity. This polymorphism is associated with obesity related heart disease.

The invention includes a method of assessing the relative susceptibility of a human to obesity, obesity related diabetes, and/or obesity related heart disease. This susceptibility can be calculated relative to a hypothetical human whose genome does not contain a single disorder-associated polymorphism in a gene associated with obesity, obesity related diabetes, and/or obesity related heart disease. Alternatively, susceptibility can be calculated relative to another human who may have one or more different disorder-associated polymorphisms than the human being assessed.

In accordance with one embodiment of the present invention, a risk score may be calculated for each of the candidate gene disease-associated risk factors, including polymorphisms. There are a number of ways of obtaining and calculating the risk score and from the risk scores calculating a susceptibility of acquiring a disease or condition.

The invention includes a method of assessing the relative susceptibility of an individual to obesity and obesity-related diseases, type 2 diabetes in particular. This susceptibility can be assessed relative to another individual whose genome does not contain a polymorphism in a candidate gene known to be associated with the disease being evaluated. The basis upon which a risk score is calculated is not critical, so long as the same basis is used for all individuals whose scores are to be compared so that risk scores can be compared to one another.

The susceptibility of an individual to obesity-related diseases provides an assessment of risks and benefits for a variety of conditions leading to obesity and obesity-related diseases and for a variety of weight loss programs. For example, some candidate-gene polymorphisms identified in the invention are associated with increased risk of obesity in individuals on a high-fat diet. Information on the susceptibility can also be used to determine the most appropriate intervention for weight loss as some of the candidate gene-polymorphisms described in the invention are known to modulate the response to exercise or diet.

Susceptibility to obesity-related diseases is assessed by determining the occurrence in an individual's genome of polymorphisms in a set of candidate genes associated with increased risk of obesity and/or obesity-related diseases and utilizing that information to obtain a risk score for each of the polymorphisms that then may be combined with risk scores for other risk factors to obtain susceptibility. Occurrence of any of the polymorphisms is an indication that the subject is more susceptible to disease (obesity or diabetes) than a subject whose genome does not comprise the polymorphism. Furthermore, occurrence of a plurality of the polymorphisms is an indication that the subject is even more susceptible to disease than a subject whose genome does not comprise the polymorphisms.

It was not previously appreciated that detection in a subject's genome of two or more polymorphisms associated with increased risk of disease in conjunction with other risk factors, individually is indicative that the subject is globally exhibiting enhanced susceptibility to disease. Previous studies have recognized only association between a polymorphism in one of these genes and a particular disease (e.g., Pro12Ala polymorphism in the PPARG gene and risk of type 2 diabetes as in Lindi et al. 2002). The inventors believe that they are the first to describe methods and kits for assessing a subject's global risk of obesity and obesity-related diseases using information from several polymorphisms simultaneously in conjunction with other risk factors.

In accordance with one embodiment of the present invention, genetic susceptibility to obesity and obesity-related diseases can be assessed by calculating a susceptibility score.

The susceptibility score can, for example, be calculated by summing, for each of the selected candidate gene polymorphisms and other risk factors, the risk scores. The risk score represents the degree to which a gene polymorphism or other risk factor is associated with the corresponding disease.

Some gene polymorphisms are strongly associated with a disease, while others have moderate effects. Several statistics can be used to assess the strength of the association between a gene polymorphism and a disease. One simple way of assessing the strength of the association is to calculate the “effect size”. The effect size is the standardized mean difference between two groups: the experimental group and the control group as shown below:

$$Effect Size = \frac{\begin{matrix}[ mean of the experimental group ] - \\ [ mean of the control group ]\end{matrix}}{standard deviation}$$

In the context of a candidate gene polymorphism, the experimental group is defined as the one carrying the mutation, while the control group is the one composed of subjects not carrying the mutation. The standard deviation is a measure of the spread of a set of values, generally those of the control group. Thus for a quantitative disease risk factor, the effect size is estimated as the mean difference between individuals homozygous or heterozygous for the mutation and those homozygous for the wild type (non mutant) allele based on data reported in published studies from the literature. For example to estimate the overall mean difference between individuals carrying the mutation and those without the mutation for an obesity-related trait like body mass index, we extract from published studies the mean and standard deviation from each genotype and each study and the overall difference is estimated as a weighted pooled mean difference Δ, as described in the formula below:

$$Δ = {\sum }_{i = 1}^{n} {w}_{i} {d}_{i} / {\sum }_{i = 1}^{n} {w}_{i} ,$$

Where n is the total number of studies, dᵢ is the difference between mean BMI of the two genotypes and wᵢ=1/Var(dᵢ) for the i^{th} study.

One interesting feature of the effect size is that it can be directly converted into statements about the overlap between the two groups in terms of percentiles. An effect size is equivalent to the “Z-score” of a normal distribution. If one goes to a normal curve table in any statistical textbook and looks up for the area under the curve associated with a z-score of 0.9, the percentage of the experimental group which exceeds the upper half of subjects from the control group may be obtained. Thus, for an effect size of 0.9, the table indicates 0.3159, which means that the average person with the mutation would score higher for the risk factor than 82% (50%+31.59%) of the subjects without the mutation. Thus the mutation would move the average subject from the 50^{th} to the 82^{th} percentile. The effect size could also be interpreted as a percent of non-overlap between the two groups. If the effect size is zero, the population distributions are superimposed on each other and there is 100% overlap or 0% non-overlap. In that case, the highest 50% of the experimental group exceeds the lowest 50% of the control group. If we define U, a measure of non-overlap, as the percentage of subjects in the experimental group that exceeds the same percentage of subjects in the control group, this will give the probability that one could guess which group a subject belongs to based on his(her) score. For a given effect size (Z score) the quantity U can be calculated as follows: U=Pz/2, where P represents the percentage of the area under the area (experimental population) falling below the Z-score. Thus, for a Z-score of 0.9, we have to look up in the Z table, the area under the curve for a Z-score of 0.45, which is 67.4% (0.50+0.1736). Thus with an effect size of 0.9, there is a 67% probability (Oust over two-thirds chance) that a subject with the mutation would be correctly identified in the high-risk group.

Another way of assessing the strength of association between a gene and a disease is to calculate the “odds ratio (OR)”, which describes the likelihood that an individual carrying the mutation will develop the disease. The OR is the equivalent of the effect size for dichotomous outcome (presence versus absence of disease). It is calculated as follows using a 2×2 table:

| | Presence of | Absence of | |
|---|---|---|---|
| | disease or | disease or | |
| | condition | condition | Total |
| Presence of | a | b | a + b |
| risk factor | | | |
| Absence of | c | d | c + d |
| risk factor | | | |

Where a, b c and d are the number of participants with each outcome in each group. From this 2×2 table, the following statistics can be calculated:

$$Risk Ratio ( RR ) = \frac{Risk of event in the risk factor group}{Risk of event in the non \text{-} risk factor groups} \begin{matrix}= & \frac{/ ( a + b )}{} \\ = & c / ( c + d )\end{matrix}$$

$$Odds Ratio ( OR ) = \frac{Odds of event in the risk factor group}{Odds of event in the non \text{-} risk factor group} \begin{matrix}= & \frac{a / b}{} & = & \frac{ad}{} \\ = & c / d & = & bc\end{matrix}$$

$$\begin{matrix}Risk difference = risk of event in the risk factor group - \\ risk of event in the control group \\ = ( a / ( a + b ) ) - ( c / ( c + d ) )\end{matrix}$$

The odds ratio is the probability that a particular event (disease) will occur to the probability that it will not. As indicated in the formula, the OR compares these probabilities in the groups with and without risk factors. An OR greater than 1.0 is an indication that the probability of disease is greater in risk factor individuals (those with the mutation) than in the non-risk factor individuals. For example, an OR of 1.50 indicates that the risk of disease is 1.5 times higher in the subjects with the mutation compared to those without it. Thus the OR reflects the strength of the association between the candidate gene polymorphism and the disease.

The risk difference is a measure of the absolute effect of the candidate gene; it describes the difference in the risk of disease between the risk factor and non-risk factor groups.

It is preferred to use the “odds ratio” (OR) or the relative risk (RR) to calculate the risk score. The relative risks are used for dichotomous traits (disease versus no disease) and are thus more appropriate to assess risk of a condition.

For each group of disease, a susceptibility score represents the subject's overall susceptibility to the disease. This susceptibility score is the sum of the risk scores associated to each candidate gene polymorphism and risk scores associated with other risk factors.

The relative susceptibility of a human to obesity, obesity related diabetes, and obesity related heart disease permits assessment of risks and benefits of a tailored diet and exercise program as intervention mechanisms In the present invention, the susceptibility of a human to obesity, type II diabetes, and obesity-related heart disease can be used to determine whether the human would benefit from a tailored diet and/or exercise program as intervention mechanism.

Although the invention is not limited to the particular disorder-associated polymorphisms in the genes identified herein, it is recognized that disorder-associated polymorphisms that occur in particular portions of the genes can be more significant indicators of obesity, type II diabetes, or obesity related heart disease than disorder-associated polymorphisms that occur in other particular portions of the genes. Thus, disorder-associated polymorphisms that occur in the previously described regions of the indicated genes can be weighted more heavily than disorder-associated polymorphisms that occur in other portions of the genes.

An important aspect of this invention is that obesity, obesity related diabetes, and obesity related heart disease can be associated with occurrence in the human's genome of a disorder-associated polymorphism in one of the genes described herein—even if there is no known biochemical or physiological association between occurrence of the polymorphism and obesity, obesity related diabetes, and/or obesity related heart disease (or incidence of) in a particular human. The present invention discloses genes and polymorphisms which are predictive indicators of the state of an individual human with respect to obesity, obesity related diabetes, and/or obesity related heart disease. By assessing whether or not disorder-associated polymorphisms occur in the genes identified herein in an individual (and how many such polymorphisms occur in those genes), one can assess an individual's risk to develop obesity, obesity related diabetes, and/or obesity related heart disease. It is to be understood that the method of the invention is applicable to essentially any disease for which a plurality of correlative genetic polymorphisms are known.

A specific example showing calculation of a susceptibility score is as follows:

Condition Selected: Obesity

Risk Factors:

Family History:

Relative to a subject without a positive family history, a subject with a family history of obesity (at least one obese parent) is about 2 times more likely to be obese.

Katzmarzyk et al, Obes. Res, 2000; Whitaker et al., New Engl. J. Med. 1997;

Risk score=2.0

Thus family history increased the risk of obesity by 100% compared to a subject without the risk factor (100×(risk score—1)%, i.e. 100%)

Physical Activity:

Physical inactivity is associated with a 2- 3-fold increased risk of obesity.

Bernstein et al., Prev.Med, 2004.

Risk score=2.0

Thus inactivity increased the risk of obesity by 100% compared to a subject without the risk factor (100×(risk score—1), i.e. 100%)

Ethnicity:

The prevalence of obesity was about 1.5 times higher in Blacks and 1.2 times higher in Hispanics compared to Whites in 2001 in the US.

Mokdad et al, JAMA, 2003; Paeratakul, et al., Int. J. Obes. 2002.

Whites: risk score 1.0

Blacks: risk score of 1.5

Hispanics: risk score of 1.2

Thus being black increases the risk by 50% compared to a white subject (100×(risk score—1)%, i.e. 50%)

Candidate Genes Effect:

ADRB3

Allison et al., (IJO, 1998) reported an effect size of 0.19 for the carriers of the Trp64 allele compared to non-carriers for BMI; this means that subjects with the mutation will score higher than 58% of the subjects without the mutation for BMI; this indicated that there is a 53% probability that subjects with the mutation would be correctly identified in the high risk group, thus an increased risk of 3%.

Risk score=1.03

LEPR

Heo et al, reported an effect size of 0.13 for the carrier of the Q223R mutation in the LEPR gene compared to non-carriers for BMI; this means that subjects with the mutation will score higher than 55% of the subjects without the mutation for BMI; this indicated that there is a 53% probability that subjects with the mutation would be correctly identified in the high risk group, thus an increased risk of 3%.

Risk score=1.03

PPARG

Masud et al., (2003) reported an effect size of 0.11 for the carriers of the Pro12Ala mutation in the PPARG gene compared to non-carriers for BMI; this means that subjects with the mutation will score higher than 54% of the subjects without the mutation for BMI; this indicated that there is a 52% probability that subjects with the mutation would be correctly identified in the high risk group, thus an increased risk of 2%.

Risk score=1.02

Thus a sedentary Black subject with a family history of obesity and carrying the 3 candidate gene mutations would have an overall susceptibility risk score of: 100%+100%+50%+3%+3%+2%=258%

An active White subject with no family history of obesity, but carrying the 3 mutations would have a susceptibility risk score of 8%.

Included in accordance with the present invention is a kit for practicing the method. The kit, at a minimum, includes materials needed to test for particular polymorphisms associated with a particular disease. The kit preferably also includes information on known risk factors and associated risk scores for the particular disease.

It will be appreciated by those skilled in the art that changes can be made to the embodiments described above without departing from the broad inventive concept thereof.

This invention is not limited to the particular embodiments disclosed, and includes modifications within the spirit and scope of the present invention as defined by the appended claims.

1. A method for assessing susceptibility of a subject to a genetically related disease or condition relative to a general population comprising:
determining the presence or absence of a plurality of selected risk factors associated with the subject and having a correlation with the disease or condition;
assigning a risk score, to each of the selected risk factors determined to be present, based upon a strength of correlation assigned to the factor with respect to the disease or condition;
combining the risk scores to calculate an overall susceptibility score, wherein the overall susceptibility score represents susceptibility of the subject to the disease or condition in relation to a base score representing the risk that a member of the general population will have the disease or condition without consideration of risk factors;
wherein the risk factors require the inclusion of at least two of age, gender, race, and family history and require the inclusion of a plurality of polymorphisms selected for known correlation with the disease or condition.

2. The method of claim 1, wherein the risk score represents the risk that a subject will have the disease or condition, when the subject also has the risk factor, divided by the risk that a subject will have the disease or condition, when the subject does not have the risk factor.

3. The method of claim 2 where the risk score is determined by a series of groups a), b), c) and d) within the general population where group a) is a group having both the risk factor and the disease or condition, group b) has the risk factor and does not have the disease or condition, group c) does not have the risk factor and has the disease or condition and group d) does not have the risk factor and does not have the disease or condition and the risk score is calculated by a risk ratio obtained from the formula [a/(a+b)][/c/(c+d)] multiplied by a constant chosen to place the risk score and base score in comparable units.

4. The method of claim 1, wherein the risk score is calculated by obtaining the standardized mean difference in the risk factors between groups a) and b), where group a) is a group carrying the polymorphism and group b) is a group not carrying the polymorphism by utilizing test results showing strength of correlation of a risk factor with the disease or condition where the test results appear in peer reviewed publications.

5. The method of claim 1 for assessing relative susceptibility of a subject to obesity, obesity related diabetes, and obesity related heart disease wherein determining the presence or absence of selected risk factors includes:
obtaining a biological sample containing genomic DNA from a subject;
testing the biological sample for nucleic acid polymorphism risk factors in one or both alleles, which polymorphisms each have a correlation with increased susceptibility to obesity, obesity related diabetes, or obesity related heart disease where the testing is for polymorphisms in at least three genes affecting the components of energy balance and in at least three genes associated with an increased risk of heart disease in overweight and obese subjects; and
assigning a risk score, to each of the selected polymorphism risk factors determined to be present, based upon a strength of correlation assigned to the factor with respect to the disease or condition.

6. The method of claim 5, wherein at least one gene is involved in regulation of appetite.

7. The method of claim 6, wherein the at least one gene is selected from the group consisting of leptin receptor gene (LEPR), dopamine receptor D2 gene (DRD2), type 2C serotonin receptor gene (HTR2C), and melanocortin-4 receptor gene (MCR4).

8. The method of claim 5, wherein at least one gene influences the capacity of fat cells to store extra energy.

9. The method of claim 8, wherein the at least one gene is selected from the group consisting of peroxisome proliferator activated receptor gamma-2 gene (PPARG), tumor necrosis factor alpha gene (TNFA), and fatty acid binding protein 2 gene (FABP2).

10. The method of claim 5, wherein at least one gene influences the amount of calories burned.

11. The method of claim 10, wherein the at least one gene is selected from the group consisting of adrenergic receptor beta-2 gene (ADRB2), adrenergic receptor beta-3 gene (ADRB3), glucocorticoid receptor gene (GRL), uncoupling protein 2 gene (UCP2) and uncoupling protein 3 gene (UCP3).

12. The method of claim 4, wherein at least one gene is associated with a risk of diabetes.

13. The method of claim 12, wherein the at least one gene is selected from the group consisting of insulin receptor substrate-1 gene (IRS1), sulfonyl urea receptor 1 gene (SUR1), and calpain 10 gene (CAPN10).

14. The method of claim 5, wherein at least one gene is associated with a risk of high blood pressure.

15. The method of claim 14, wherein the at least one gene is selected from the group consisting of angiotensin converting enzyme gene (ACE) and angiotensinogen gene (AGT).

16. The method of claim 5, wherein at least one gene is associated with a risk of high blood cholesterol.

17. The method of claim 16, wherein the at least one gene is selected from the group consisting of apolipoprotein E gene (APOE), apolipoprotein B gene (APOE), and lipoprotein lipase gene (LPL).

18. The method of claim 5, wherein testing comprises testing for a nucleic acid polymorphism in one or both alleles of at least two genes selected from the group consisting of leptin receptor gene, dopamine receptor D2 gene, type 2C serotonin receptor gene, melanocortin-4 receptor gene, peroxisome proliferator activated receptor gamma-2 gene, tumor necrosis factor alpha gene, fatty acid binding protein 2 gene, adrenergic receptor beta-2 gene, adrenergic receptor beta-3 gene, glucocorticoid receptor gene, uncoupling protein 2 gene, uncoupling protein 3 gene, insulin receptor substrate-1 gene, sulfonyl urea receptor 1 gene, calpain 10 gene, angiotensin converting enzyme gene, angiotensinogen gene, apolipoprotein E gene, apolipoprotein B gene, and lipoprotein lipase gene.

19. The method of claim 5, wherein the nucleotide polymorphism is selected from the group consisting of a polymorphism manifested as a change from a glutamine residue to an arginine residue at amino acid residue 223 in leptin receptor protein encoded by exon 6 of leptin receptor gene (LEPR), a polymorphism manifested as a change from a lysine residue to an arginine residue at amino acid residue 109 in leptin receptor protein encoded by leptin receptor gene (LEPR), a polymorphism manifested as a change from a lysine residue to an asparagine residue at amino acid residue 656 in leptin receptor protein encoded by leptin receptor gene (LEPR), a polymorphism manifested as a change from a serine residue to a serine residue at amino acid residue 343 in leptin receptor protein encoded by leptin receptor gene (LEPR) containing an altered codon, a polymorphism manifested as a change from a serine residue to a cysteine residue at amino acid residue 311 in dopamine receptor protein encoded by dopamine receptor D2 gene (DRD2), a polymorphism manifested at the Taq1A marker of dopamine receptor D2 gene (DRD2), a polymorphism manifested as a change from a serine residue to a cysteine residue at amino acid residue 282 in dopamine receptor D2 protein encoded by dopamine receptor D2 gene (DRD2), a polymorphism manifested at a NcoI RFLP (C→T exon 6) of dopamine receptor D2 gene (DRD2), a polymorphism manifested as a change from a proline residue to a serine residue at amino acid residue 310 in dopamine receptor D2 protein encoded by dopamine receptor D2 gene (DRD2), a polymorphism manifested as a change from a nucleotide alanine to a nucleotide guanine position demonstrated by SNP rs1124491(A/G) of dopamine receptor D2 gene (DRD2), a polymorphism manifested as a change from a cysteine residue to a serine residue at amino acid residue 23 of type 2C serotonin receptor protein encoded by type 2C serotonin receptor gene (HTR2C), a polymorphism manifested as a change from a leucine residue to a valine residue at amino acid residue 4 of type 2C serotonin receptor protein encoded by type 2C serotonin receptor gene (HTR2C), a polymorphism manifested as a change from a serine residue to a isoleucine residue at amino acid residue 169 of melanocortin-4 receptor protein encoded by melanocortin-4 receptor gene (MC4R), a polymorphism manifested as a change from a isoleucine residue to a valine residue at amino acid residue 103 of melanocortin-4 receptor protein encoded by melanocortin-4 receptor gene (MC4R), a polymorphism manifested as a change from an arginine residue to a glycine residue at amino acid residue 98 of melanocortin-4 receptor protein encoded by melanocortin-4 receptor gene (MC4R), a polymorphism manifested as a change from a proline residue to an alanine residue at amino acid residue 12 of peroxisome proliferator activated receptor gamma-2 protein encoded by peroxisome proliferator activated receptor gamma-2 gene (PPARG), a polymorphism manifested as a change from a proline residue to an alanine residue at amino acid residue 40 of peroxisome proliferator activated receptor gamma-2 protein encoded by peroxisome proliferator activated receptor gamma-2 gene (PPARG), a polymorphism manifested as a change from a proline residue to a glutamine residue at amino acid residue 83 of melanocortin-4 receptor protein encoded by melanocortin-4 receptor gene (MC4R), move upwards with the other MC4R polymorphisms a polymorphism manifested at position −308 changing a nucleotide guanine for a nucleotide alanine of tumor necrosis factor alpha gene (TNFA), a polymorphism manifested as a change from a histidine residue to an asparagine residue at amino acid residue 52 of tumor necrosis factor alpha protein encoded by tumor necrosis factor alpha gene (TNFA), a polymorphism manifested as a change from a proline residue to a leucine residue at amino acid residue 84 of tumor necrosis factor alpha gene (TNFA), a polymorphism manifested as a change from an alanine residue to a threonine residue at amino acid residue 54 of fatty acid binding protein 2 encoded by fatty acid binding protein 2 gene (FABP2), a polymorphism manifested as a change from a threonine residue to an alanine residue at amino acid residue 55 of fatty acid binding protein 2 encoded by fatty acid binding protein 2 gene (FABP2), a polymorphism demonstrated as SNP rs1511025, a polymorphism manifested as a change from a glycine residue to an arginine residue at amino acid residue 16 of adrenergic receptor beta-2 protein encoded by adrenergic receptor beta-2 gene (ADBR2), a polymorphism manifested as a change from a glutamine residue to a glutamic acid residue at amino acid residue 27 of adrenergic receptor beta-2 protein encoded by adrenergic receptor beta-2 gene (ADBR2), a polymorphism manifested as a change from a threonine residue to an isoleucine residue at amino acid residue 164 of adrenergic receptor beta-2 protein encoded by adrenergic receptor beta-2 gene (ADBR2), a polymorphism manifested as a change from a serine residue to a cysteine residue at amino acid residue 220 of adrenergic receptor beta-2 protein encoded by adrenergic receptor beta-2 gene (ADBR2), a polymorphism manifested as a change from a tryptophan residue to an arginine residue at amino acid residue 64 of adrenergic receptor beta-3 protein encoded by adrenergic receptor beta-3 gene (ADRB3), a polymorphism manifested as a change from a threonine residue to a methionine residue at amino acid residue 265 of adrenergic receptor beta-3 protein encoded by adrenergic receptor beta-3 gene (ADRB3), a polymorphism manifested as a change from an asparagine residue to a serine residue at amino acid residue 363 of corticoid receptor protein encoded by corticoid receptor gene (GRL), a polymorphism manifested as a change from a phenylalanine residue to a valine residue at amino acid residue 65 of corticoid receptor protein encoded by corticoid receptor gene (GRL), a polymorphism manifested at position +647 of corticoid receptor protein encoded by corticoid receptor gene (GRL), a polymorphism manifested as a change from an alanine residue to a valine residue at amino acid residue 55 of uncoupling protein 2 encoded by uncoupling protein 2 gene (UCP2), a polymorphism manifested as a change from a nucleotide cytosine residue to a nucleotide thymine residue at position −55 of uncoupling protein 3 encoded by uncoupling protein 3 gene (UCP3), a polymorphism manifested as a change from an arginine residue to a cysteine residue at amino acid residue 282 of uncoupling protein 3 encoded by uncoupling protein 3 gene (UCP3), a polymorphism manifested as a change from a valine residue to a isoleucine residue at amino acid residue 102 of uncoupling protein 3 encoded by uncoupling protein 3 gene (UCP3), a polymorphism manifested as a change from a tyrosine residue to a tyrosine residue at amino acid residue 99 of uncoupling protein 3 encoded by uncoupling protein 3 gene (UCP3)containing an altered codon, a polymorphism manifested as a change from a methionine residue to a threonine residue at amino acid residue 209 of insulin receptor substrate-1 protein encoded by insulin receptor substrate-1 gene (IRS1), a polymorphism manifested as a change from a threonine residue to a threonine residue at amino acid residue 759 of sulfonyl urea receptor 1 protein encoded by sulfonyl urea receptor 1 gene (SUR1) containing an altered codon, a polymorphism manifested as a change from an alanine residue to a serine residue at amino acid residue 1369 of sulfonyl urea receptor 1 protein encoded by sulfonyl urea receptor 1 gene (SUR1), a polymorphism manifested as UCSNP-43 (g.4852 G/A) of CAPN10, a polymorphism manifested as UCSNP-44 (g.4841 T/C) of calpain 10 gene (CAPN10), a polymorphism manifested as a change from a threonine residue to an alanine residue at amino acid residue 504 of calpain 10 protein encoded by calpain 10 gene (CAPN10), a polymorphism manifested as an ACE I/D polymorphism of angiotensin converting enzyme gene (ACE), a polymorphism manifested as a change from an arginine residue to a serine residue at amino acid residue 1286 of angiotensin converting enzyme encoded by angiotensin converting enzyme gene (ACE), a polymorphism manifested as a change from a methionine residue to a threonine residue at amino acid residue 235 of angiotesinogen protein encoded by angiotensinogen gene (AGT), a polymorphism manifested as a change from a threonine residue to a methionine residue at amino acid residue 174 of angiotesinogen protein encoded by angiotensinogen gene (AGT), a polymorphism manifested by the isoforms ApoeE2, ApoeE3, or ApoeE4 of apolipoprotein E gene (APOE), a polymorphism manifested as a change from a cysteine residue to an arginine residue at amino acid residue 130 of apolipoprotein E encoded by apolipoprtein E gene (APOE), a polymorphism manifested by the EcoRI locus of apolipoprotein B gene (APOB), a polymorphism manifested as a change from an aspartic acid residue to an asparagine residue at amino acid residue 9 of lipoprotein lipase encoded by lipoprotein lipase gene (LPL), and a polymorphism manifested as a truncated lipoprotein lipase at amino acid residue 446 due to a change from a serine code for amino acid residue 447 to a stop codon in lipoprotein lipase gene (LPL).

20. The method of claim 5, wherein each polymorphism is a single nucleotide polymorphism, a sequence tagged site, a restriction site polymorphism, or a restriction fragment length polymorphism.

21. The method of claim 4 wherein the disease or condition is selected from the group consisting of obesity, obesity related diabetes, and obesity related heart disease.

22. The method of claim 21 wherein the overall susceptibility score represents genetic susceptibility to one or more of obesity, obesity related diabetes, and obesity related heart disease.

23. The method according to claim 5, wherein the biological sample is blood, hair, mucosal scrapings, semen, tissue biopsy, or saliva.

24. The method according to claim 5, wherein the subject is a mammal.

25. The method according to claim 24, wherein the mammal is a human.

26. The method of claim 5 where the disease or condition is obesity related diabetes and the biological sample is tested for a nucleic acid polymorphism in one or both alleles in at least three genes associated with an increased risk of obesity related diabetes.

27. The method of claim 26, wherein the at least three genes are selected from the group consisting of peroxisome proliferator activated receptor gamma-2 gene, tumor necrosis factor alpha gene, fatty acid binding protein 2 gene, uncoupling protein 2 gene, insulin receptor substrate-1 gene, sulfonyl urea receptor 1 gene, calpain 10 gene, and angiotensin converting enzyme gene.

28. The method of claim 27, wherein the nucleotide polymorphism is selected from the group consisting of a polymorphism manifested as a change from a proline residue to an alanine residue at amino acid residue 12 of peroxisome proliferator activated receptor gamma-2protein encoded by peroxisome proliferator activated receptor gamma-2 gene (PPARG), a polymorphism manifested as a change from a proline residue to an alanine residue at amino acid residue 40 of peroxisome proliferator activated receptor gamma-2 protein encoded by peroxisome proliferator activated receptor gamma-2 gene (PPARG), a polymorphism manifested at position −308 changing a nucleotide guanine for a nucleotide alanine of tumor necrosis factor alpha gene (TNFA), a polymorphism manifested as a change from a histidine residue to an asparagine residue at amino acid residue 52 of tumor necrosis factor alpha protein encoded by tumor necrosis factor alpha gene (TNFA), a polymorphism manifested as a change from a proline residue to a leucine residue at amino acid residue 84 of tumor necrosis factor alpha protein encoded by tumor necrosis factor alpha gene (TNFA), a polymorphism manifested as a change from an alanine residue to a threonine residue at amino acid residue 54 of fatty acid binding protein 2 encoded by fatty acid binding protein 2 gene (FABP2), a polymorphism manifested as a change from a threonine residue to an alanine residue at amino acid residue 55 of fatty acid binding protein 2 encoded by fatty acid binding protein 2 gene (FABP2), a polymorphism demonstrated as SNP rs1511025, a polymorphism manifested as a change from an alanine residue to a valine residue at amino acid residue 55 of uncoupling protein 2 encoded by uncoupling protein 2 gene (UCP2), a polymorphism manifested as a change from a methionine residue to a threonine residue at amino acid residue 209 of insulin receptor substate-1 protein encoded by insulin receptor substrate-1 gene (IRS1), a polymorphism manifested as a change from a threonine residue to a threonine residue at amino acid residue 759 of sulfonyl urea receptor 1 protein encoded by sulfonyl urea receptor 1 gene (SUR1) containing an altered codon, a polymorphism manifested as a change from a alanine residue to a serine residue at amino acid residue 1369 of sulfonyl urea receptor 1 protein encoded by sulfonyl urea receptor 1 gene (SUR1), a polymorphism manifested as UCSNP-43 (g.4852 G/A) of calpain 10 gene (CAPN10), a polymorphism manifested as UC SNP-44 (g.4841 T/C) of calpain 10 gene (CAPN10), a polymorphism manifested as a change from a threonine residue to an alanine residue at amino acid residue 504 of calpain 10 protein encoded by calpain 10 gene (CAPN10), a polymorphism manifested as an insertion or a deletion, known as the ACE I/D polymorphism of angiotensin converting enzyme gene (ACE), and a polymorphism manifested as a change from an arginine residue to a serine residue at amino acid residue 1286 of angiotensin converting enzyme encoded by angiotensin converting enzyme gene (ACE).

29. The method of claim 5 where the disease or condition is obesity related heart disease and the sample is tested for nucleic acid polymorphisms in at least genes associated with an increased risk of heart disease.

30. The method of claim 29, wherein the at least three genes are selected from the group consisting of peroxisome proliferator activated receptor gamma-2 gene, tumor necrosis factor alpha gene, fatty acid binding protein 2 gene, adrenergic receptor beta-2 gene, adrenergic receptor beta-3 gene, uncoupling protein 2 gene, insulin receptor substrate-1 gene, sulfonyl urea receptor 1 gene, calpain 10 gene, angiotensin converting enzyme gene, angiotensinogen gene, apolipoprotein E gene, apolipoprotein B gene, and lipoprotein lipase gene.

31. The method of claim 30, wherein the nucleotide polymorphism is selected from the group consisting of a polymorphism manifested as a change from a proline residue to an alanine residue at amino acid residue 12 of peroxisome proliferator activated receptor gamma-2 protein encoded by peroxisome proliferator activated receptor gamma-2 gene (PPARG), a polymorphism manifested as a change from a proline residue to an alanine residue at amino acid residue 40 of peroxisome proliferator activated receptor gamma-2 protein encoded by peroxisome proliferator activated receptor gamma-2 gene (PPARG), a polymorphism manifested at position −308 changing a nucleotide guanine for a nucleotide alanine of tumor necrosis factor alpha gene (TNFA), a polymorphism manifested as a change from a histidine residue to an asparagine residue at amino acid residue 52 of tumor necrosis factor alpha protein encoded by tumor necrosis factor alpha gene (TNFA), a polymorphism manifested as a change from a proline residue to a leucine residue at amino acid residue 84 of tumor necrosis factor alpha protein encoded by tumor necrosis factor alpha gene (TNFA), a polymorphism manifested as a change from an alanine residue to a threonine residue at amino acid residue 54 of fatty acid binding protein 2 encoded by fatty acid binding protein 2 gene (FABP2), a polymorphism manifested as a change from a threonine residue to an alanine residue at amino acid residue 55 of fatty acid binding protein 2 encoded by fatty acid binding protein 2 gene (FABP2), a polymorphism demonstrated as SNP rs1511025, a polymorphism manifested as a change from a glycine residue to an arginine residue at amino acid residue 16 of adrenergic receptor beta-2 protein encoded by adrenergic receptor beta-2 gene (ADBR2), a polymorphism manifested as a change from a glutamine residue to a glutamic acid residue at amino acid residue 27 of adrenergic receptor beta-2 protein encoded by adrenergic receptor beta-2 gene (ADBR2), a polymorphism manifested as a change from a threonine residue to an isoleucine residue at amino acid residue 164 of adrenergic receptor beta-2 protein encoded by adrenergic receptor beta-2 gene (ADBR2), a polymorphism manifested as a change from a serine residue to a cysteine residue at amino acid residue 220 of adrenergic receptor beta-2 protein encoded by adrenergic receptor beta-2 gene (ADBR2), a polymorphism manifested as a change from a tryptophan residue to an arginine residue at amino acid residue 64 of adrenergic receptor beta-3 protein encoded by adrenergic receptor beta-3 gene (ADBR3), a polymorphism manifested as a change from a threonine residue to a methionine residue at amino acid residue 265 of adrenergic receptor beta-3 protein encoded by adrenergic receptor beta-3 gene (ADBR3), a polymorphism manifested as a change from an alanine residue to a valine residue at amino acid residue 55 of uncoupling protein 2 encoded by uncoupling protein 2 gene (UCP2), a polymorphism manifested as a change from a methionine residue to a threonine residue at amino acid residue 209 of insuline receptor substrate-1 protein encoded by insuline receptor substrate-1 gene (IRS1), a polymorphism manifested as a change from a threonine residue to a threonine residue at amino acid residue 759 of sulfonyl urea receptor 1 protein encoded by sulfonyl urea receptor 1 gene (SUR1), a polymorphism manifested as a change from an alanine residue to a serine residue at amino acid residue 1369 of sulfonyl urea receptor 1 protein encoded by sulfonyl urea receptor 1 gene (SUR1), a polymorphism manifested as UCSNP-43 (g.4852 G/A) of calpain 10 gene (CAPN10), a polymorphism manifested as UCSNP-44 (g.4841 T/C) of calpain 10 gene (CAPN10), a polymorphism manifested as a change from a threonine residue to an alanine residue at amino acid residue 504 of calpain 10 protein encoded by calpain 10 gene (CAPN10)CAPN10, a polymorphism manifested as an ACE I/D polymorphism of angiotensin converting enzyme gene (ACE), a polymorphism manifested as a change from an arginine residue to a serine residue at amino acid residue 1286 of angiotensin converting enzyme encoded by angiotensin converting enzyme gene (ACE), a polymorphism manifested as a change from a methionine residue to a threonine residue at amino acid residue 235 of angiotensinogen protein encoded by angiotensinogen gene (AGT), a polymorphism manifested as a change from a threonine residue to a methionine residue at amino acid residue 174 of angiotensinogen protein encoded by angiotensinogen gene (AGT), a polymorphism manifested by the isoforms ApoeE2, ApoeE3, or ApoeE4 of apolipoprotein E protein encoded by apolipoprotein E gene (APOE), a polymorphism manifested as a change from a cysteine residue to an arginine residue at amino acid residue 130 of apolipoprotein E protein encoded by apolipoprotein E gene (APOE), a polymorphism manifested by the EcoRI locus of apolipoprotein B protein encoded by apolipoprotein B gene (APOB), a polymorphism manifested as a change from an aspartic acid residue to an asparagine residue at amino acid residue 9 of lipoprotein lipase encoded by lipoprotein lipase gene (LPL), and a polymorphism manifested as a truncated lipoprotein lipase at amino acid residue 446 due to a change from a serine code for amino acid residue 447 to a stop codon in lipoprotein lipase gene (LPL).

32. The method of claim 5 wherein the risk factors include all of age, gender, race, family history and a plurality of polymorphisms selected for known correlation with the disease or condition.

33. A kit for practicing the method of claim 1.

34. A kit for practicing the method of claim 2.

35. A kit for practicing the method of claim 3.

36. A kit for practicing the method of claim 4.

37. A kit for practicing the method of claim 5.
